(19) United States
(12) Patent Application Publication
(10) Pub. No.: US 2003/0215534 A1
(43) Pub. Date: Nov. 20, 2003

(54) Extraction of unmilled psyllium

(75) Inventors: Paul Alfred Cimiluca, Cincinnati, OH (US); John Philip Hecht, West Chester, OH (US); Theresa Marie Kajs, Loveland, OH (US); Graham John Myatt, Bracknell (GB); Jason Allen Stamper, Covington, KY (US); Michael Anthony Staun, Loveland, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/368,226

(22) Filed: Feb. 18, 2003

Related U.S. Application Data
(60) Provisional application No. 60/381,972, filed on May 20, 2002.

(51) Int. Cl. A61K 35/78

(52) U.S. Cl. 424/776; 424/725

(57) ABSTRACT

Methods of fractionating unmilled psyllium seed husk to obtain a compressible, gel-forming polysaccharide with a reduced gelation rate and improved swell volume. The gel-forming fraction isolated by the present invention is useful for administration to humans to normalize bowel function, reduce human serum cholesterol levels, and regulate blood glucose levels. The gel-forming polysaccharide also exhibits reduced allergenicity as compared to psyllium seed husk.

### CROSS REFERENCE TO PRIORITY APPLICATION

This application claims priority under Title 35, United States Code 119(e) from Provisional Application Serial No. 60/381,972, filed May 20, 2002.

### FIELD OF THE INVENTION

The present invention relates to a method of fractionating unmilled psyllium to yield a gel-forming polysaccharide suitable for administration to humans to normalize bowel function, provide Taxation, reduce human serum cholesterol levels, and regulate blood glucose levels.

### BACKGROUND OF THE INVENTION

Psyllium seed husk containing products are currently widely used for normalizing bowel function and Taxation. It has also been shown that psyllium seed husk is effective for reducing human serum cholesterol levels and in controlling blood glucose levels in diabetics.

These benefits are typically achieved by ingestion of psyllium seed husk, which is obtained from the seed coat from plants of the Plantago genus. To render a laxative effect, a typical human dose of psyllium seed husk is from about 3 grams to about 20 grams, taken from about 1 to about 3 times per day. In order to administer such a large amount of psyllium seed husk, the husk is often milled or ground and subsequently dispersed in water or an aqueous beverage for consumption by the user (for example, METAMUCIL®, sold by The Procter & Gamble Company). In addition to milling, typically, sanitization of the psyllium seed husk is performed prior to any further processing, in order to reduce microbial contamination of the psyllium seed husk. This sanitization step can be costly and difficult to perform.

Psyllium seed husk contains natural mucilage, forming a gelatinous mass on contact with water. Thus, milled psyllium seed husk, with its increased surface area, exhibits very poor disperability and mixability in water as the particles tend to agglomerate. Hydration takes place over the surface of the agglomerated aggregates to form gel-coated lumps, the interiors of which are still substantially dry. These lumps are extremely difficult to disperse. Various methods have been employed to improve the dispersability of milled psyllium husk in an aqueous medium. For example, U.S. Pat. No. 5,425,945 to Barbera, discloses a drink mix composition comprising agglomerated psyllium seed husk with an edible acid uniformly dispersed throughout the agglomerating coating to obtain improved mixability and dispersability.

However, once dispersed in an aqueous solution, the agglomerated psyllium husk quickly begins to hydrate and gel with an accompanying increase in the viscosity of the drink solution. Again, various methods have been employed to reduce this gelation rate and provide an aesthetically pleasing product. U.S. Pat. No. 5,356,618, to Daggy et al., teaches that the addition of calcium citrate malate to a composition comprising milled psyllium seed husk results in a reduced gelation rate of the husk when mixed with an aqueous solution. However, despite these improvements, the consumer of the psyllium seed husk suspension typically drinks the liquid in a relatively short period of time (less than about two minutes) in order to avoid having to drink an aesthetically unpleasant, high viscosity liquid.

Sanitized, milled psyllium seed husk has been incorporated in baked items, such as cookies, crackers and similar food items to render solid dosage forms. However, the fast gelation of the psyllium husk is noticeable in these preparations as well. Psyllium husk containing preparations have a tendency to begin to gel in the mouth during consumption, resulting in an unpleasant mouthfeel and poor aesthetics. It is generally necessary to consume such baked items with significant amounts of water or another beverage for ease of swallowing. In addition, such solid psyllium seed husk preparations must be large in size or, alternatively, multiple preparations must be consumed in order to deliver an effective amount of psyllium seed husk. Therefore, a psyllium containing composition that is convenient, easily administered and has acceptable aesthetics and good mouthfeel characteristics is still needed.

Previously the focus has been to provide a swallowable psyllium-containing tablet with acceptable dissolution properties, thus avoiding problems of poor mouthfeel. U.S. Pat. No. 4,999,200, to Casillan, teaches a swallowable psyllium-containing tablet comprising psyllium, a binder, a wetting agent and a disintegrating agent. Unfortunately, swallowable psyllium tablets, while convenient, often have poor dissolution properties. Like the powdered drink mix, once introduced into an aqueous environment hydration takes place over the surface of the pill, creating a gel coating, the interiors of the pill remain substantially dry. For swallowable pills this can lead to incomplete dissolution in the gastrointestinal tract. Therefore, there is a need to provide a psyllium-containing dosage form, suitable for chewing, where the chewing action disintegrates the tablet into smaller, discrete particles prior to swallowing but which undergoes minimal gelling in the mouth, and has acceptable mouthfeel and good aesthetics as perceived by the consumer.

Methods of fractionating psyllium seed husk into various polysaccharide components are known. These fractions of psyllium seed husk deliver the same therapeutic benefits as psyllium seed husk and can act as a suitable substitute for psyllium seed husk in various dosage forms. For example, U.S. Pat. No. 6,287,609 to Marlett et al., teaches a multiple extraction process for obtaining three distinct fractions from milled psyllium husk, including an alkali insoluble fraction (Fraction A), an alkali soluble/acid gel-forming fraction (Fraction B), and an acid soluble fraction (Fraction C). The alkali soluble/acid gel-forming fraction has a slower rate of gelation than non-fractionated psyllium seed husk. It has been surprisingly discovered that fractionation of unmilled psyllium seed husk provides an improved gel-forming material with increased swell volume over the fractions obtained by the Marlett method. Multiple extraction steps can be employed in the fractionation. However, a single extraction step renders a high yield of a gel-forming polysaccharide with reduced rates of gelation, and an increased swell volume, suitable for oral administration. Where raw (unsanitized) psyllium seed husk is used as the starting material, the extraction is followed by a disinfection step. The resulting gel-forming polysaccharide has a slower rate of gelation and reduced allergenicity over sanitized, milled psyllium seed husk.

It has also been discovered that fluidized bed drying the gel-forming polysaccharide obtained by the present method provides a compressible composition, suitable for tabletting by direct compression.

### SUMMARY OF THE INVENTION

The present invention relates to methods of fractionating psyllium seed husks to obtain a gel-forming polysaccharide. Unmilled psyllium husk serves as the starting material for the fractionation, which provides a gel-forming polysaccharide suitable for use in oral dosage forms administered for normalizing bowel function, reducing human serum cholesterol levels, and regulating blood glucose levels. The gel-forming polysaccharide formed by the present method has a slow gelation rate and increased swell volume in comparison to non-fractionated psyllium seed husk and psyllium fractions obtained by the prior art. Where only a single extraction (alkaline solubilization) step is conducted, the yield of gel-forming polysaccharide is improved. In addition, the gel-forming polysaccharide isolated by the present invention has reduced allergenicity relative to sanitized, milled psyllium seed husk.

Unmilled psyllium seed husks are mixed in an aqueous alkaline solution comprising from about 0.1M to about 1.0M hydroxyl ions. The psyllium seed husk may be previously sanitized or unsanitized. The alkaline environment serves to fractionate the husks into an alkali insoluble fraction and an alkali soluble fraction. Where unsanitized psyllium seed husk is employed, subsequent disinfection of the mixture reduces microbial contamination to acceptable levels. The alkali insoluble fraction is then removed by any means known in the art. Acidification of the alkaline solution yields a gel material, i.e. the gel-forming polysaccharide. The resulting mixture may be separated in a second extraction step, or alternatively, directly dewatered and dried. Drying the gel material renders a powdered gel-forming polysaccharide useful for further processing, such as tabletting. Where the gel material is dried by the fluidized bed drying method, the resulting gel-forming polysaccharide powder is directly compressible.

All documents cited are, in relevant part, incorporated herein by reference; the citation of any document is not to be construed as an admission that it is prior art with respect to the present invention.

### DETAILED DESCRIPTION OF THE INVENTION

Definitions

By “safe and effective amount”, as used herein, is meant an amount of an active agent (e.g. the gel-forming polysaccharide) high enough to significantly improve the condition to be treated, but low enough to avoid serious side effects (at a reasonable benefit/risk ratio), within the scope of sound medical judgment. The “safe and effective amount” may vary with the particular condition being treated, the age and physical condition of the patient being treated, the severity of the condition, the duration of treatment, the nature of concurrent therapy, the specific form of the source employed, and the particular vehicle from which the agent is applied.

The term “oral composition” as used herein means any pharmaceutical composition intended to be administered to the stomach of a mammal via the mouth of said mammal.

The term “solid oral dosage form” refers to a physically discrete unit suitable as a unitary dosage for human subjects and other mammals, each containing a predetermined quantity of active material (e.g. the gel-forming polysaccharide) calculated to produce the desired therapeutic effect. Solid oral dosage forms that are suitable for the present compositions include tablets, pills, capsules, lozenges, chewable tablets, troches, cachets, pellets and the like. In one embodiment the compositions of the present invention are in the form of chewable tablets containing particles or granules of the gel-forming polysaccharide.

For purposes herein, the term “raw” refers to psyllium seed husk that has not been sanitized (prior to the initial alkaline solubilization step), by any method known in the art, such as by steam sanitization.

The term “compressible” as used herein refers to granules or powders that are capable of undergoing compaction, reversible deformation and finally irreversible deformation as applied stress increases, ultimately resulting in a reduction in volume. Compressible powders or granules may be compressed into tablet form.

The term “direct compression” refers to the process of compressing tablets directly from powdered material. Direct compression is appropriate where the physical nature of the powdered material need not be modified prior to tabletting.

The term “swell volume” as used herein is the volume of gel mass formed when the gel-forming polysaccharide, equivalent to 0.5 grams psyllium on a weight basis (or 0.5 grams psyllium seed husk) are combined with water to a total volume of 100 mL (milliliters) in a cylinder at ambient temperature. The cylinder is inverted several times at the start of the test to insure thorough mixing, as well as at 4 hours and 8 hours from the start of the test. The swell volume is recorded 24 hours after the start of the test. Swell volume provides a measure of the ability of the gel-forming polysaccharide (or psyllium) to absorb water. The swell volume is reported in milliliters of swelled gel forming polysaccharide mass per grams of dry gel forming polysaccharide.

Percentages and ratios herein are by weight of total composition, unless otherwise indicated.

The method of the present invention relates to fractionating psyllium seed husk to obtain a gel-forming polysaccharide fraction of psyllium seed husk. To obtain the desired fraction the following steps are employed:

a) mixing unmilled psyllium seed husks in an aqueous alkaline solution comprising from about 0.1M to about 1.0M hydroxyl ions, thereby fractionating the husks into a mixture comprising an alkali insoluble fraction and an alkali soluble gel fraction;

b) where the psyllium seed husk has not been sanitized prior to alkaline solubilization, the alkali insoluble and alkali soluble fractions are disinfected by any means known in the art such as pasteurization, irradiation, electron beam or pulsed light;

c) removing the alkali insoluble fraction; and

d) acidifying the alkaline solution to reveal an acid gel-forming material.

Acidification may be followed by dewatering of the acidified gel material with a dessicant and subsequent drying of the gel material.

In one embodiment, the method of fractionating psyllium seed husk comprises the following steps:

Step 1. Suspending unmilled psyllium seed husk in a dilute alkaline aqueous solution containing a reducing agent.

Step 2. Where previously unsanitized psyllium is utilized, disinfecting the alkali soluble and alkali insoluble fractions, by any means known in the art such as pasteurization, irradiation, electron beam or pulsed light.

Step 3. Removing the alkali insoluble material by any process known in the art, for example centrifugation, filtration, expression or settling.

Step 4. Acidifying the solution to a pH of about 4.5 to about 6.5 by the addition of acid, to yield an acid gel-forming material, i.e. the gel-forming polysaccharide.

Step 5. Dewatering the gel material by the addition of a desiccant with high shear mixing and then separating the gel material from the desiccant/water solution.

Step 6. Extruding the gel material into individual particles with an average particle size of greater than 250 microns.

Step 7. Fluidized bed drying the gel material rendering the compressible gel-forming polysaccharide in powder form.

The starting material employed in the method of the present invention is generally unmilled psyllium seed husk. That is, the psyllium seed husk need not be milled or physically altered or refined, prior to the initial alkaline solubilization step. U.S. Pat. No. 6,287,609 to Marlett et al., teaches that it is necessary for the psyllium seed husk to be processed so that it is in small pieces, prior to alkaline solubilization, for ease of separation of the viscous polysaccharides from the insoluble fibers of the psyllium husk.

However, clumping and agglomeration of the milled psyllium seed husk occurs when the milled husk is added to the alkaline mixture. It has been discovered that the use of unmilled psyllium seed husk as an initial starting material avoids clumping or agglomerating of the psyllium material during mixing with the alkaline solution, but does not hinder the effectiveness of the alkaline solubilization step. The use of unmilled psyllium as a starting material for the fractionation provides a gel-forming polysaccharide with increased swell volume. The swell volume of the gel-forming polysaccharide obtained by the present invention is greater than about 40 milliliters of gel per 0.5 grams dry gel-forming polysaccharide, in one embodiment greater than about 50 milliliters of gel per 0.5 grams dry gel-forming polysaccharide. The percent yield of the gel-forming polysaccharide of the present invention is at least about 75%, in one embodiment at least about 80%. The psyllium seed husk of the present invention may or may not be sanitized prior to processing. Where raw (unsanitized) psyllium is employed in the present method, a disinfection step is incorporated in the present method and may be carried out as described below.

Alkaline solubilization (Step 1) of psyllium seed husk is known. Typically, previous alkaline solubilization processes utilized concentrations of strong bases and lacked the presence of a reducing agent. Recognizing the harsh nature of this treatment and the partial degradation of polysaccharide chains in the gel-forming fraction, it has been shown that a gel-forming fraction of psyllium husk could be obtained, presumably in a form more suitable for further fractionation, if desired, using a much less concentrated alkaline solution and a suitable reducing agent, such as borohydride. Though up to about 4N alkaline solution can be utilized, the concentration of base in the alkaline solubilization is at least about 0.1N and not more than about 1.0N; in one embodiment at least about 0.1N and not more than about 0.5N; and in yet another embodiment at least about 0.1N and not more than about 0.3N. Any standard base can be used in the alkaline extraction, including, but not limited to, sodium hydroxide, potassium hydroxide, lithium hydroxide, ammonium hydroxide, and tetramethyl ammonium hydroxide. A suitable ratio of psyllium seed husks to alkaline solution is from about 0.1 gram seed husk to about 400 ml (milliliters) of alkaline solution to about 4 grams seed husk to about 400 ml alkaline solution. The alkaline solubilization should be carried out at a pH of from about 9 to about 12.

A chemical reducing agent, such as borohydride, should be added to the alkaline solubilization step to minimize base-catalyzed depolymerization. Borohydrides suitable for this step include, but are not limited to, lithium borohydride, potassium borohydride and sodium cyanoborohydride. In one embodiment the reducing agent is sodium borohydride. An effective concentration of a reducing agent is from about 50 mg/L (milligrams/liter) to about 10 g/L (grams/liter), in one embodiment from about 100 mg/L to about 4 g/L, in another embodiment from about 500 mg/L to about 2 g/L, and in yet another embodiment from about 800 mg/L to about 1.2 g/L.

The time of solubilization can be varied from about 15 minutes to about 24 hours, in one embodiment from about 30 minutes to about 180 minutes, for optimum efficiency.

Likewise, the temperature at which the solubilization step is conducted can vary from about 5° C. to about 40° C. In one embodiment the time of solubilization is from about 60 minutes to about 120 minutes at ambient temperature. The alkaline solubilization may optionally be carried out in a nitrogen atmosphere to prevent oxidation from occurring.

The disinfecting step, Step 2, is required when the psyllium seed husk has not been sanitized prior to mixing with the alkaline solution. If the unmilled psyllium seed husk is sanitized by any method known in the art, such as steam sanitation, prior to the alkaline solubilization step, this disinfection step is not necessary. Disinfection refers to inactivating, destroying, eliminating, or inhibiting the growth of microorganisms. In one embodiment these microorganisms are disease-producing agents. Disinfection of the combined alkali soluble and alkali insoluble fractions may be conducted by any means known in the art. For example, pasteurization, irradiation, electron beam and pulsed light are all acceptable means of disinfecting the alkali soluble and alkali insoluble fraction mixture. In one embodiment, the mixture is pasteurized. Pasteurization entails heating the mixture to a moderate temperature for a period of time to disinfect, without changing, to any extent, the chemical composition of the mixture. Pasteurization may be carried out at a temperature of from about 90° C. to about 120° C. for a period of from about 30 seconds to about 120 seconds.

The alkali insoluble material is separated from the alkali soluble materials in Step 3 of the instant method. This can be accomplished by any separation means known in the art that will not alter substantially the insoluble material, for example centrifugation. One skilled in the art will know how to alter the time and force of the centrifugation to adapt the separation to different centrifuge rotors, plant materials and alkaline solutions. Other methods to accomplish this separation are well known in the art and may be better suited for large-scale production of the gel-forming polysaccharide, such as settling, filtration, or expression. Optionally, the insoluble material can be further washed with the alkaline solution and re-separated in an effort to improve the yield of the alkaline soluble material.

In Step 4 of the instant method, the alkaline soluble materials are acidified to a pH of from about 4.5 to about 6.5, in one embodiment from about 5 to about 6, to yield an acid gel-forming material, i.e. the gel-forming polysaccharide. Suitable acids for acidification include, but are not limited to, acetic, hydrochloric, sulfuric, oxalic, trichloroacetic and trifluoroacetic acids. The duration and temperature of the acidification can vary. The acidification may suitably take place at ambient temperature for about 2 hours, though the time and temperature may vary.

Optionally, a second extraction may be appropriate at this stage of the fractionation process. Where desired, the acid soluble and acid gel-forming fractions may be separated, by any means known in the art, such as centrifugation, settling, straining and the like. Again an optional washing with water, buffer, or other suitable solvent may be employed to improve the efficiency of the separation. This second extraction may be employed to deliver a more purified gel-forming polysaccharide, but may also lead to degradation and loss of some of the gel-forming polysaccharide. It has been found that multiple extraction steps are not necessary to yield a suitable gel-forming polysaccharide with increased swell volume and a reduced gelation rate.

Excess water is then removed from the acid gel-forming polysaccharide fraction in Step 5 of the present method. Any method known in the art may be used to dewater the gel fraction. In one embodiment the gel material may be dewatered by desiccation with a solvent, such as ethanol, acetone, methanol or isopropyl alcohol. The addition of the solvent may occur with high shear mixing. The gel material is then separated from the solvent/water mixture by any method known in the art. For ease and simplicity of drying, the solids content of the gel material should be at least about 50%, in one embodiment the solids content is at least about 75%, in another embodiment the solids content of the gel material is about 80%.

The gel material may be dried in any manner known in the art, such as lyophilization, fluidized bed drying or vacuum tray drying. In one embodiment, fluidized bed drying of the gelatinous material is employed. The gel material is extruded to form small grain-like particles and placed into a fluidized bed dryer. The particle size of the gel-forming polysaccharide should be greater than 250 microns, in one embodiment from about 250 microns to about 1000 microns, and in another embodiment from about 350 to about 750 microns. The fluidized bed dryer may be equipped to provide a cyclonic airflow, which helps prevent the particles sticking together and allows the particles to fluidize. The extruded particles are suspended in the column of air until dried to at least about 85% solids content. During drying, the gel material should be maintained at a temperature of less than about 75° C. It is preferred that the solids content of the gel material is greater than about 20% prior to fluidized bed drying. If necessary, previously dried gel material may be added by mixing to the low solids content gel material, prior to fluidized bed drying, to increase the solids content to greater than about 20%. Not intending to be bound by theory, it is believed that the fluidized bed drying technique renders a gel-forming polysaccharide powder composition wherein the individual particles retain a honeycomb shape. The honeycomb shape is useful to facilitate compression of the gel-forming polysaccharide powder, particularly by direct compression means, into a solid dosage form.

The gel-forming polysaccharide obtained by the method disclosed herein is comprised primarily of xylose and arabinose. In one embodiment, the gel-forming polysaccharide has at least about 50% xylose and arabinose by weight, in another embodiment at least about 75% xylose and arabinose, in yet another embodiment at least about 80% xylose and arabinose. In one embodiment, the xylose to arabinose dry weight ratio is at least about 3:1, in one embodiment from about 3:1 to about 4.5:1, in another embodiment from about 3:1 to about 4:1 and in yet another embodiment from about 3.3:1 to about 3.6:1. In one embodiment the gel-forming polysaccharide comprises from about 55% to about 70% of xylose and from about 15% to about 20% of arabinose. In addition, low levels of galactose and uronic acid are present in the gel-forming polysaccharide of the present invention. Generally the level of galactose is less than about 2%, in one embodiment from about 1% to about 2%. The level of uronic acid is generally less than 10%. In one embodiment the dry weight ratio of xylose to galactose is more than about 25:1, in another embodiment more than about 30:1 and in yet another embodiment more than about 35:1. In one embodiment the dry weight ratio of xylose to uronic acid is more than about 5:1, in one embodiment about 10:1 and in yet another embodiment about 15:1. Generally, the gel-forming polysaccharide has the following sugar composition:

| Component | Amount present in gel-forming polysaccharide |
|---|---|
| Xylose | From about 55% to about 70% |
| Arabinose | From about 15% to about 20% |
| Rhamnose | From 0% to about 5% |
| Mannose | From 0% to about 0.5% |
| Galactose | From about 1% to about 2% |
| Glucose | From 0% to about 0.5% |
| Uronic Acid | From about 0.5% to about 50% |

Importantly, the fractionation and isolation of the gel-forming polysaccharide by the present invention produces a gel-forming polysaccharide, which has reduced allergenicity when compared to milled, sanitized psyllium seed husk. As used herein the term “allergenicity” is a measure of the amount of allergenic protein present in the gel-forming polysaccharide. Psyllium seed husk contains specific protein fractions, which are considered allergens. Allergenicity is determined by extracting proteins from a sample of material (e.g. the gel-forming polysaccharide or psyllium seed husk) and then determining the allergenicity of those proteins by known electrophoresis techniques, such as sodium dodecylsulfate polyacrylamide gel electrophoresis (SDS-PAGE), or immunoblotting. One skilled in the art can readily utilize these techniques to evaluate the reduction in allergenicity of a material versus a control (e.g. psyllium seed husk). For example, U.S. Pat. No. 5,248,502, to Ndife, teaches that immunoblotting is used to determine the extent of IgE antibody binding to specific psyllium proteins, providing a measure of the allergenicity of psyllium protein fractions. The gel-forming polysaccharide of psyllium husk obtained by the method disclosed herein has reduced allergenicity in comparison to milled sanitized psyllium seed husk (the control). A reduction in allergenicity of greater than about 90% versus the control, in one embodiment greater than about 95% versus the control, is achieved by fractionating psyllium seed husk by the present method. Thus, the level of allergenic protein present in the gel-forming polysaccharide is less than about 10% of the allergenic protein present in psyllium seed husk, in one embodiment less than about 5% of the allergenic protein present in psyllium seed husk. Not intending to be bound by theory, it is believed that the reduction in allergencity is due to several factors. Allergenic proteins are believed to be mainly present in the alkali insoluble fraction, which is removed in large part during Step 3 of the present method. The subsequent dewatering of the remaining gel material with a solvent/dessicant may result in denaturing of the proteins remaining in the gel material thereby further reducing allergenicity.

Methods of Use

The gel-forming polysaccharide is useful for the treatment of gastrointestinal disorders. The material can be used alone or in combination with other active substances, in a safe and effective amount, for the treatment of constipation and laxation and for normalizing bowel function. The gel-forming polysaccharide may also be effective for providing more complete evacuation of the bowel and thereby rendering a detoxifying effect. In addition, the gel-forming polysaccharide is useful for reducing human serum cholesterol and controlling blood glucose levels in diabetics and may be used alone or in conjunction with other actives substances.

The gel-forming polysaccharide may be incorporated in pills, capsules, and other solid dosage forms known in the art. The gel-forming polysaccharide may also be useful in oral liquid compositions. Alternatively, the gel material may be utilized in dried, powdered form and incorporated into various food products. In one embodiment the compositions may be tabletted for use as a swallowable or chewable tablet. The gel material isolated by the present method may be directly compressed into solid oral dosage forms suitable for chewing, such as chewable tablets, for consumption by the consumer. Each tablet may comprise from about 100 mg to about 5000 mg of the gel-forming material, in one embodiment, a chewable tablet may comprise from about 1000 mg to about 1500 mg of the gel-forming material. The gel-forming polysaccharide should be administered at a level of at least about 2 grams, from about 1 to about 3 times per day to achieve the desired laxative or normalizing benefits or relieving constipation.

### EXAMPLES

### Example 1

### Fractionation of Psyllium Seed Husk

Raw, unmilled psyllium seed husk (2 grams) is stirred with 0.2N sodium hydroxide (400 milliliters) containing sodium borohydride (400 milligrams) in a nitrogen atmosphere at ambient temperature for 90 minutes. The pH of the solution is from 10 to 11. The solution is passed through a pasteurizer at a temperature of 100° C. for a period of 50 seconds. Once pasteurized, the mixture is centrifuged for 20 minutes at 23,500×g. The supernatant is decanted from an insoluble fraction that settles out in the centrifuge bottle. The insoluble fraction is mixed with fresh sodium hydroxide/sodium borohydride solution (100 milliliters) and recentrifuged for 15 minutes to increase yield of the soluble fraction. The pH of the supernatant is adjusted to 5.5 by the addition of acetic acid at ambient temperature with stirring, forming a gel. The gel is desiccated with isopropanol added with high shear mixing. The isopropanol solution is then decanted from the gel. The solids content of the gel is 30%. The gel material is passed through an extruder and extruded into individual particles with an average particle size of 500 microns. The extruded particles enter a fluidized bed dryer fitted with a Conidur screen. The air temperature is maintained at 80° C. The gel temperature remains below 70° C. throughout the drying process. The particles are dried to a powder, with 90% of the water being removed. The yield of the gel-forming polysaccharide is 85%.

### Example 2

### Fractionation of Psyllium Seed Husk

Unmilled psyllium seed husk is mixed with an alkaline solution and acidified as above. The gel-forming polysaccharide is further separated from the acid soluble materials by centrifugation for 20 minutes at 23,500×g. The gel-forming polysaccharide and the acid soluble material are then each separately desiccated and dried in the manner described in Example 1.

### Example 3

### Fractionation of Psyllium Seed Husk

Raw, unmilled psyllium seed husk (4 grams) is stirred with 0.2N sodium hydroxide (400 milliliters) containing sodium borohydride (400 milligrams) in a nitrogen atmosphere at ambient temperature for 90 minutes. The pH of the solution is from 10 to 11. The solution is passed pasteurized at a temperature of 110° C. for a period of 40 seconds. Once pasteurized, the mixture is centrifuged for 20 minutes at 23,500×g. The supernatant is decanted from an insoluble fraction that settles out in the centrifuge bottle. The pH of the supernatant is adjusted to 5.5 by the addition of acetic acid at ambient temperature with stirring, forming a gel. The gel is desiccated with ethanol added with high shear mixing. The ethanol solution is then decanted from the gel. The solids content of the gel is 15%. Previously obtained dry gel-forming polysaccharide powder is added to the composition to increase the solids content to 25%. The gel material is passed through an extruder and extruded into individual particles with an average particle size of 500 microns. The extruded particles enter a fluidized bed dryer fitted with a cyclonic airflow screen, such as a Conidur screen. The air temperature is maintained at 80° C. The gel temperature remains below 70° C. throughout the drying process. The particles are dried to a powder, 90% of the water being removed.

### Example 4

### Chewable Tablet Comprising the Gel-Forming Polysaccharide

Chewable tablets, total weight 2.5 grams, are manufactured in the following manner: Where optional ingredients are desired, a pre-mix is prepared comprising a flavorant, a colorant and citric acid. The gel-forming polysaccharide, prepared in the manner described in Example 1, is dry blended with sorbitol for 10 minutes, each component having an average particle size of about 500 microns. The pre-mix, if desired, is added and blended for an additional 10 minutes. Magnesium stearate is added and the composition is blended for another 5 minutes. The mixture is directly compressed into tablets using pressures of from 2000 psi to 4000 psi. The final composition comprises the following components by weight:

| | Example | Example | Example | Example |
|---|---|---|---|---|
| Component | 1A | 1B | 1C | 1D |
| Gel-Forming | 50.0% | 50.0% | 50.0% | 50.0% |
| Polysaccharide | | | | |
| Sorbitol (Neosorb | 48.16% | 47.95% | 47.75% | 50.0% |
| P20/60) | | | | |
| Magnesium Stearate | 0.5% | 0.25% | 0.4% | |
| Flavorant | 0.4% | 0.6% | 0.6% | |
| Colorant | 0.14% | 0.2% | | |
| Citric Acid | 0.8% | 1% | 1.25% | |

It is understood that the examples and embodiments described herein are for illustrative purposes only and that various modifications or changes in light thereof will be suggested to one of skill in the art without departing from the scope of the present invention.

What is claimed:

1. A method of fractionating psyllium seed husks to obtain a gel-forming polysaccharide comprising the steps of:
a) mixing raw, unmilled psyllium seed husks in an aqueous alkaline solution comprising from about 0.1M to about 1.0M hydroxyl ions, thereby fractionating the husks into a mixture comprising an alkali insoluble fraction and an alkali soluble gel fraction;
b) disinfecting the alkali soluble and alkali insoluble fractions;
c) removing the alkali insoluble fraction; and
d) acidifying the alkaline solution, yielding the gel-forming polysaccharide.

2. The method of claim 1, wherein the gel-forming polysaccharide comprises xylose and arabinose in a dry weight ratio of at least about 3:1.

3. The method of claim 2 wherein the gel-forming polysaccharide further comprises 1% to 2% of galactose.

4. The method of claim 3 wherein the gel-forming polysaccharide comprises from about 55% to about 70% xylose.

5. The method of claim 4 wherein the gel-forming polysaccharide comprises from about 15% to about 20% arabinose.

6. The method of claim 5 wherein the gel-forming polysaccharide has a swell volume of at least about 50 millilters gel per 0.5 grams of dry gel-forming polysaccharide.

7. The method of claim 1 wherein the step of disinfecting the alkali soluble and alkali insoluble fractions comprises the step of pasteurizing the alkali soluble and alkali insoluble fractions.

8. The method of claim 1 wherein the alkaline solution is acidified to a pH of about 3 to about 6.

9. The method of claim 8 further comprising the step of dewatering the gel-forming polysaccharide.

10. The method of claim 9 wherein the gel-forming polysaccharide has a level of allergenic protein that is less than about 10% of that present in psyllium seed husk.

11. The method of claim 10 wherein the gel-forming polysaccharide has a level of allergenic protein that is less than about 5% of that present in psyllium seed husk.

12. The method of claim 9 further comprising the step of drying the gel-forming polysaccharide.

13. The method of claim 12 wherein the drying step comprises the steps of extruding the acidic gel-forming polysaccharide into discrete particles and drying the discrete particles by fluidized bed drying.

14. The method of claim 13 wherein the gel-forming polysaccharide is compressible.

15. The method of claim 14 wherein the average particle size of the discrete particles of the gel-forming polysaccharide is at least about 250 microns.

16. The method of claim 15 wherein the average particle size of the discrete particles of the gel-forming polysaccharide is from about 250 to about 1000 microns.

17. The method of claim 1 wherein the percent yield of the gel-forming polysaccharide produced is at least about 75%.

18. The method of claim 17 wherein the percent yield of the gel-forming polysaccharide produced is at least about 80%.

19. A method of fractionating psyllium seed husks to obtain a compressible gel-forming polysaccharide comprising the steps of:
a) mixing raw, unmilled psyllium seed husks in an aqueous alkaline solution comprising from about 0.1M to about 1.0M hydroxyl ions, thereby fractionating the husks into a mixture of an alkali insoluble fraction and an alkali soluble gel fraction;
b) disinfecting the mixture;
c) removing the alkali insoluble fraction;
d) acidifying the alkaline solution to a pH of about 3 to about 6 to yield the gel-forming polysaccharide;
e) dewatering the gel-forming polysaccharide;
f) extruding the gel forming polysaccharide into discrete particles; and
g) fluidized bed drying the gel-forming polysaccharide.

20. A gel-forming polysaccharide produced by the method of claim 21.

21. The gel-forming polysaccharide of claim 20 wherein the level of allergenic protein is less than about 10% of that present in psyllium seed husk.

22. The gel-forming polysaccharide of claim 21 wherein the level of allergenic protein is less than about 5% of that present in psyllium seed husk.

23. A method of fractionating psyllium seed husks to obtain a gel-forming polysaccharide comprising the steps of:
a) mixing raw, umnilled psyllium seed husks in an aqueous alkaline solution comprising from about 0.1M to about 1.0M hydroxyl ions, thereby fractionating the husks into a mixture comprising an alkali insoluble fraction and an alkali soluble gel fraction;
b) disinfecting the mixture;
c) removing the alkali insoluble fraction;
d) acidifying the alkaline solution to yield the gel-forming polysaccharide; and
e) separating the gel-forming polysaccharide from the acid solution.

24. The method of claim 23, wherein the gel-forming polysaccharide fraction comprises xylose and arabinose in a dry weight ratio of at least about 3:1.

25. The method of claim 24 wherein the gel-forming polysaccharide further comprises 1% to 2% of galactose.

26. The method of claim 25 wherein the gel-forming polysaccharide comprises from about 55% to about 70% xylose.

27. The method of claim 26 wherein the gel-forming polysaccharide comprises from about 15% to about 20% arabinose.

28. The method of claim 27 wherein the gel-forming fraction has a swell volume of at least about 55 milliliters gel per 0.5 grams of dry gel-forming polysaccharide.

29. The method of claim 1 wherein the alkaline solution is acidified to a pH of about 3 to about 6.

30. The method of claim 29 further comprising the step of dewatering the gel-forming polysaccharide.

31. The method of claim 30 further comprising the step of drying the gel-forming polysaccharide.

32. A method of fractionating psyllium seed husks to obtain a gel-forming polysaccharide comprising the steps of:
a) mixing sanitized, unmilled psyllium seed husks in an aqueous alkaline solution comprising from about 0.1M to about 1.0M hydroxyl ions, thereby fractionating the husks into a mixture comprising an alkali insoluble fraction and an alkali soluble gel fraction;
b) removing the alkali insoluble fraction; and
c) acidifying the alkaline solution to yield the gel-forming polysaccharide.
f) separating the gel-forming polysaccharide from the acid solution.

33. The method of claim 32, wherein the gel-forming polysaccharide fraction comprises xylose and arabinose in a dry weight ratio of at least about 3:1.

34. The method of claim 33 wherein the gel-forming polysaccharide comprises from about 55% to about 70% xylose and from about 15% to about 20% arabinose.

35. The method of claim 34 wherein the gel-forming polysaccharide has a swell volume of at least about 55 milliliters gel per 0.5 grams of dry gel-forming polysaccharide.

36. The method of claim 32 wherein the alkaline solution is acidified to a pH of about 3 to about 6.

37. The method of claim 36 further comprising the step of dewatering the acidic gel-forming polysaccharide.

38. The method of claim 37 further comprising the step of drying the acidic gel-forming polysaccharide.

39. A method of drying a gel-forming polysaccharide obtained from fractionation of psyllium seed husk, comprising the steps of:
a) extruding the gel forming polysaccharide into discrete particles; and
b) fluidized bed drying the gel forming polysaccharide.
wherein the dried gel-forming polysaccharide is compressible.

40. The method of claim 39 wherein the average particle size of the discrete particles of the gel-forming polysaccharide is at least about 250 microns.
